(19) United States
(12) Patent Application Publication
(10) Pub. No.: US 2011/0014700 A1
(43) Pub. Date: Jan. 20, 2011

(54) Methods of Storing and Providing Samples of Cells and Tissue

(76) Inventor: Herbert Zech, Bregenz (AT)

Correspondence Address: FISH & ASSOCIATES, PC;ROBERT D. FISH, 2603 Main Street, Suite 1000, Irvine, CA, 92614-6232, US

(21) Appl. No.: 12/744,445

(22) PCT Filed: Nov. 25, 2008
(86) PCT No.: PCT/EP08/66173

§ 371 Date: Jul. 21, 2010

(30) Foreign Application Priority Data
Nov. 25, 2007 (EP) 07121472.0

(51) Int. Cl. C12N 5/07

(52) U.S. Cl. 435/374

(57) ABSTRACT

A cell or tissue sample from a donor is stored in bank wherein the donor can initially determine the sample status to (a) exclusively retain, (b) make public, or (c) optionally make public his cells or tissue. Where the donor opts for optional public storage, the donor may in response to a third party request convert the optional public storage status to public status or private status top thereby share or exclusively retain his cells or tissue. Most preferably, depending on the initial and/or subsequent choice by the donor, an incentive or disincentive is provided.

### FIELD OF THE INVENTION

The field of the invention is methods of storing and providing samples of cells or tissue.

### BACKGROUND

Stem cells obtained from bone marrow or umbilical cord blood are increasingly used in the treatment of various disorders.

Bone marrow transplantation is primarily used in current treatments of various disorders such as cancer, autoimmune diseases, blood disorders, and metabolic disorders. These treatments take advantage of stem cells present in the bone marrow. However, the number of compatible bone marrow donors is typically limited, and finding a suitable donor often takes several weeks or even months.

Umbilical cord blood has been used as an alternative to bone marrow as a source of stem cells in the treatment of these disorders. Umbilical blood is newborn blood collected from the umbilical cord after severing of the cord from the newborn. Private umbilical cord blood banks have been established in several countries where cord blood collected at birth is typically cryopreserved for long-term storage and deposited in a cord blood depository. The deposited cord blood is used should the donor subsequently develop a condition that can be treated by implantation of stem cells derived from his cord blood. A person whose cord blood was deposited in a bank at birth can thus be assured of a compatible source of stem cells, and is not dependent upon finding a suitable donor. However, private banking precludes the public from having access to HLA-matched or otherwise compatible cells.

Thus, there is still a need for improved systems and methods of storing and providing samples of cells or tissue.

### SUMMARY OF THE INVENTION

It turned out that various cells or tissue, in particular stem cells, allow treatment of numerous disorders. Particular useful cells are for example cells of bone marrow, cells of umbilical cord blood, or amniotic fluid cells. Particular useful tissues are, for example, adipose tissues or cells isolated from the adipose tissue. Other body tissue, body substances, or organs may be useful such as blood, foreskin, or other skin, fetal stem cells, adult stem cells, germ cells, or germ stem cells. An increasing number of cells or tissue seems allowing treatment of numerous disorders. Even though the high potential of such cells or tissue for treating disorders is more and more recognized, only a very limited number of such treatments are accomplished.

The underlying problem of the present invention is therefore to provide methods that allow increasing the number of such treatments. This problem is solved with a method of storing and providing samples of cells or tissue. The method of the present invention allows attracting more donors to store samples of cells or tissue, and therefore allows building of a large bank of samples of cells or tissue. The method according to the present invention also motivates the donors to provide the samples to a third party requestor. The method of the present invention therefore allows building of a bank with a huge amount of samples that are accessible in a public registry and that can be provided for third party requestors. Only such a huge amount of samples available in the bank provides a fair chance that a third party requestor may find a sample of a compatible donor. One advantage of the present invention is therefore that the chance of the bank to provide a sample of a compatible donor is increased, therefore allowing to increase the number of treatments using cells or tissue, most preferably using stem cells. It has also been found out that a compatible bone marrow donor can only be found for a certain number of all cases, the number varying also according to the ethnic background and may be very low for certain races requiring samples of cells or tissue. The method according to the invention therefore raises the chance to find a suitable donor also for individuals of such ethnic or otherwise limited background.

The present invention provides a method of storing a sample of cells or tissue in a cell or tissue bank, in which the following steps are included:

a) receiving the sample from a donor, and b) associating the sample in response to a first donor instruction with one of a private registry, a public registry, or a public optional registry; and if the donor opts for the public optional registry c) receiving from the donor, in response to a third party request for the sample, a second instruction to either switch association from the public optional registry to the private registry or to allow providing the sample to the third party requestor. Most preferably the following steps are also included: d) wherein the donor is provided an incentive when the sample is provided to the third party requestor; and e) wherein the donor is provided a disincentive when the donor switches association from the public optional registry to the private registry.

The method according to the invention has various advantages. The most preferred method comprises providing an incentive. The donor becomes aware that a sample of cells or tissue has a certain value because he may receive an incentive when the sample is provided to a third party requestor. The donor therefore becomes aware that the donated cells or tissue is something valuable, which motivates the donor not to throw away the cells, for example the umbilical cord, but to store them. The donor may opt whether the sample shall be associated in a public registry or in a private registry only. Most importantly, after a third party request for the donor's sample, the donor may voluntarily decide whether he would like to exclusively retain the sample for his own use in the future by transferring the sample into a private registry and by paying a fee, or whether he would like to release the sample and provide the sample to the third party requestor, thereby receiving an incentive. In contrast, there are known non-profit public banks, which serve the public interest by providing samples to people in need, and in such public banks, the donor has after donation no more rights to the donated cells or tissue as anybody else and the bank decides on its own about who may benefit from and receive the sample. It should therefore be recognized that the method according to the invention creates a win-win-situation for the donor, in that, after storing the sample in the bank, the donor has sometime in the future the choice of receiving an incentive by releasing the sample, or has the choice of keeping the sample for his own benefit in the future.

One purpose or benefit of the method according to the invention is therefore to motivate donors to store samples of cells or tissue, and to decide later on whether an individual donor prefers keeping the samples for his own purpose, or whether the individual donor prefers to release the sample, thereby helping a third party and receiving an incentive. Whichever option the donor chooses, he or she will receive a benefit. One purpose of the invention is therefore to serve best the donors needs, to either store the sample for his or her own exclusive benefit, or if such exclusive storage is not desired, to release the sample for the benefit of a third party. It is further assumed that an increasing number of techniques will become available which will allow in vitro expanding of cells in the sample, which enables the donor to provide one part of the sample to the third party requestor, and to keep another part of the sample for his own. In another advantageous method step the received sample is subdivided into at least two parts wherein one part of the subdivided sample is associated to the private registry, and therefore accessible for the donor only, whereby another part of the subdivided sample is associated to the public registry or the public optional registry. In vitro expanding of the cells of the private registry may be sufficient to provide the donor with an appropriate amount of cells or tissue.

As contemplated methods are very attractive, the methods allow building of a cell or tissue bank with a very large amount of samples, thus increasing the chance that the cell or tissue bank contains a sample matching the specific needs of a third party requestor. Therefore there is a fairly high chance to be able to provide a reasonable or even large number of third party requestors with samples, which allows generating a continuous and reasonable cash flow by selling the samples to the third party requestors. This in turn allows charging a donor with a very moderate fee for storing his sample in the cell or tissue bank, so that an increasing number of donors can afford to store their cells or tissue in a bank. Beside the method of storing the sample, also various financing models will be disclosed in the following examples.

Various objects, features, aspects and advantages of the inventive subject matter will become more apparent from the following detailed description of preferred embodiments, along with the accompanying drawings in which like numerals represent like components.

### BRIEF DESCRIPTION OF THE DRAWING

FIG. 1 is a flowchart for a method of obtaining cells or tissue from donors, for storing obtained cells or tissue, and for providing cells or tissue;

FIG. 2 is a flowchart of a third party request to obtain cells or tissue;

FIG. 3 is one exemplary diagram of various sample statuses;

FIG. 4 is another exemplary diagram of various sample statuses;

FIG. 5 shows an exemplary system for providing samples of cells or tissue in accordance with one embodiment of the invention.

### DETAILED DESCRIPTION

In the following description, the invention is described by a method of storing of umbilical cord blood of a donor in a cord blood depository or cord blood bank. This is by way of example only, it being understood that the invention may be implemented for the provision of any cell, body tissue or organ in a depository or bank of such cells, tissues or organs.

As used herein, the term “donor” refers to an individual who has deposited a cell, body tissue or organ in a bank of such cells, tissues or organs, or a guardian or other party authorized to act on behalf of the individual.

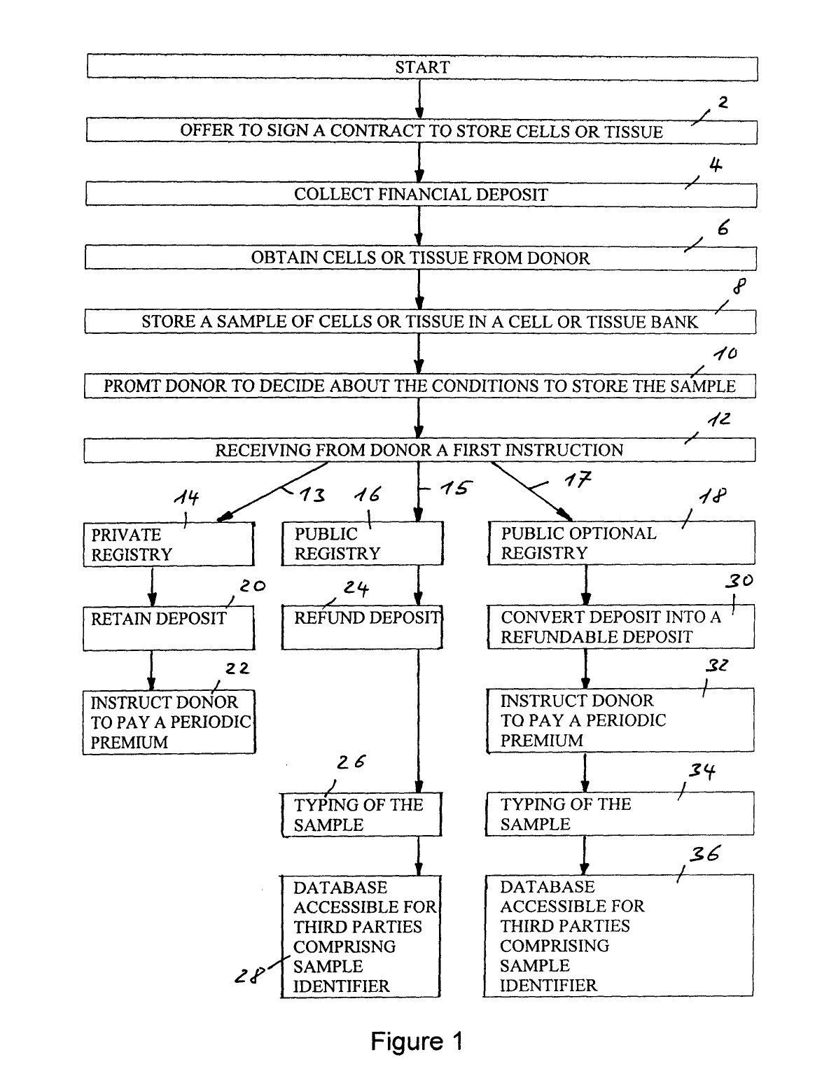

FIG. 1 shows a flow chart for carrying out one embodiment of the method of the invention. In step 2 the donor is offered a contract to sign for storing umbilical cord blood. In step 4 a financial deposit is collected. In step 6 umbilical cord blood of a donor is obtained and, in step 8, the obtained cord blood is stored in an umbilical cord blood bank. Details relating to the donor and the stored blood are entered into a database. Together with step 2, or after one of steps 4, 6, or 8, the donor is in step 10 asked to decide about the conditions to store the at least one sample, whereby the donor has the options of storing the sample in a private registry 14 (via 13), a public registry 16 (via 15) or a public optional registry 18 (via 17). After receiving from the donor the first instruction in step 12, the database is updated according to the option selected by the donor to store the sample. Storing the sample in the private registry 14 has the effect that the sample is stored exclusively for the donor, so that only he is allowed to decide about the use the sample. Whereas storing the sample in the public registry 16 has the effect that the sample is donated to the bank and therefore is available for a third party requestor without reverting back to the donor. Attributes of the samples belonging to the public registry 16 are preferably accessible in public databases, so that a third party seeking for a sample may find and order it. A third option is storing the sample in a public optional registry 18. Attributes of the samples belonging to the public optional registry 18 are also accessible to third parties, but if a third party intends to order the sample, the donor is first asked to decide whether he would like to give away the sample, or whether be would like to switch the status of his sample to the private registry 14, so that only the donor will have access to the sample in the future. In another advantageous method, the method step 8 comprises subdividing the received sample into at least two parts and associating one part of the subdivided sample to the private registry and associating at least another part to the public or public optional registry.

In a preferred method, also the way of handling the collected financial deposit is influenced by selecting one of private registry 14, public registry 16 and public optional registry 18. FIG. 1 shows one exemplary advantageous method about how to handle the collected financial deposit. If the option “private registry” 14 is selected, in step 20 the deposit collected in step 4 is retained. In a further preferred step 22, the donor is also instructed to pay a periodic premium to keep the sample in the status private registry 14. If the option “public registry” 16 is selected, the effect is that the sample is donated to the bank and the deposit collected in step 4 is refunded in step 24 to the donor. If the option “public optional registry” 18 is chosen, the deposit collected in step 4 is in step 30 converted into a refundable deposit. In a further advantageous step 32 the donor is instructed to pay a periodic premium.

A step of typing the sample is necessary at least for samples having the status “public registry” 16 or “public optional registry” 18. The typing of the sample can take place some when after obtaining the cell from the donor in step 6. Most preferably the first donor instruction 12 is requested after the step 6 of obtaining the sample and before a step 26, 34 of typing the sample, in particular of human leukocyte antigen (HLA) typing.

FIG. 1 discloses an example of a method wherein the sample having the status public registry 16 is typed in step 26 and the characteristics of the sample are in step 28 stored in a database accessible for third parties, the sample comprising an identifier. Further, the sample having the status public optional registry 18 is typed in step 34, and the characteristics of the sample are stored in step 36 in a database accessible for third parties, the sample comprising an identifier.

In a preferred method, step 12 is requested within four weeks after receiving the sample. This relatively long period of four weeks to decide is of particular importance if umbilical cord blood is stored. The parents receiving a baby and having stored the cord blood of their baby in the bank should have no time pressure to decide about which of the options 14, 16, and 18 they would like to select.

The step 4 of collecting the financial deposit may preferably be completed between step 2 and steps 20, 24, and 30 respectively. When it is intended to store umbilical cord blood, step 4 is most preferably completed several days or weeks before birth of the baby.

The following describes methods of procedures where a third party requests a sample. The method of matching a request with the sample available in the bank is not described because this does not form part of the invention. Depending on whether the sample has the status “public” 16 or “public optional” 18, different procedures apply. If the sample is of the public registry 16, the sample is delivered to the third party, preferably after the third party paying a fee.

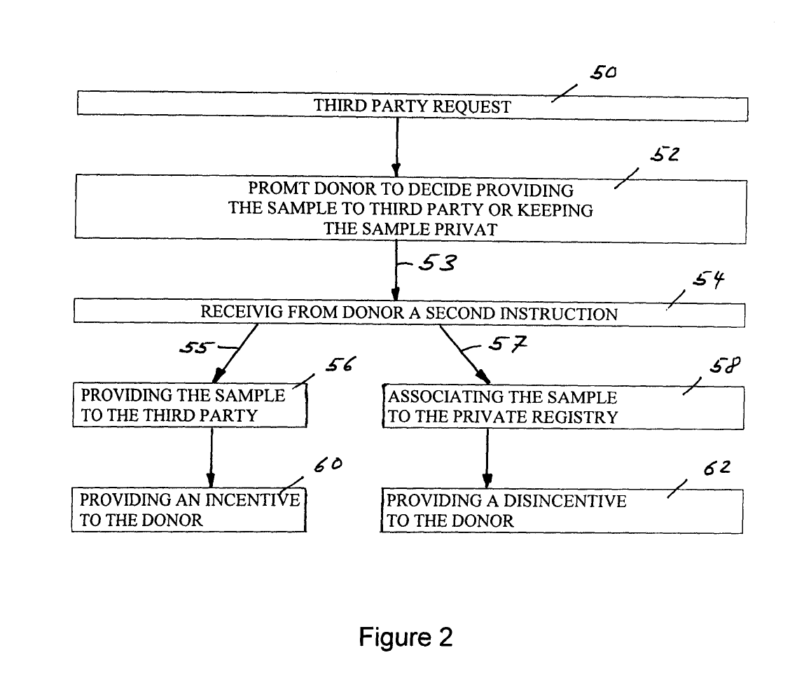

FIG. 2 shows a flow chart for carrying out a third party request 50 for a sample having the status “public optional”. If the third party requests a sample of the public optional registry, the donor is requested in step 52 to decide whether the sample is given to the third party, or whether the status of the sample is switched to the status “private registry”. After a second instruction of the donor has been received in step 54 (via 53; the first instruction of the donor has been received in step 12 of FIG. 1), the sample is, according to the donor's instruction, either provided to the third party in step 56 (via 55), or the sample is associated to the private registry in step 58 (via 57). In the most preferred embodiment, an incentive is provided to the donor in step 60, after deciding to provide the sample to a third party. Preferably at least the refundable deposit is returned to the donor, optionally an interest-bearing deposit, and most preferably an additional incentive such as an amount of money. In the most preferred embodiment, a disincentive is provided to the donor in step 62, after deciding to associate the sample to the private registry 14, for example by conversion of the financial deposit into a non-refundable fee. Step 60, providing an incentive and/or step 62, providing a disincentive, may also be omitted.

The method disclosed in FIG. 2 has the advantage, that this procedure allows a large amount of donors to offer their sample to third parties, whereby the donor has the advantage to decide at the moment a third party requests the sample to either receive an incentive when the sample is provided to a third party, or to accept a disincentive to switch the status of the sample to private registry. The methods disclosed in FIGS. 1 and 2 allow building of a bank very fast, with a large number of samples accessible to third parties. The methods disclosed in FIGS. 1 and 2 also allow showing that umbilical cells are valuable cells, because the donor has the option, after a third party request, to get his deposit refunded, and in addition, to receive an incentive, which might be for example an amount of money. It is therefore readily apparent for everybody that the cells must be something valuable. The method disclosed in FIGS. 1 and 2 therefore motivates many people to store cells, tissues or organs in a bank.

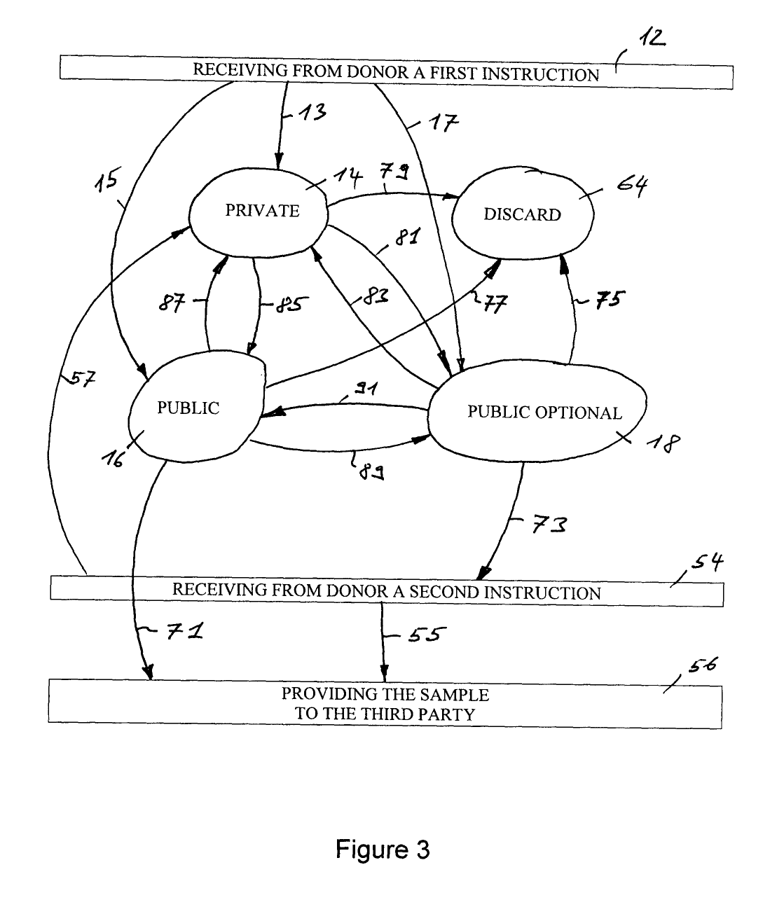

FIG. 3 shows the various statuses a sample may have in the method according to the invention. As also shown in FIG. 1, in step 12 of receiving from the donor a first instruction it is decided to which of the statuses “private” 14, “public” 16 and “public optional” 18 the sample will be assigned to (via 13, 15, and 17, respectively). After a third party request and the selected sample having the status “public” 16, the sample is, as indicated by arrow 71, provided to the third party in step 56. After a third party request and the selected sample having the status “public optional” 18, the method shown in FIG. 2 applies. Depending on the second instruction 54 received from the donor, the sample having the status “public optional” will, as indicated by arrows 73, 55, and 57, either be provided to the third party in step 56, or the sample will have the status “private” 14. Independent of a third party request, the status of the sample may also change for other reasons. It might be necessary, for whatever reason, to discard the sample in step 64, as indicated by arrows 75, 77, and 79. It also may happen that the donor, for whatever reasons decides to switch the status of the sample form one of “private” 14, “public” 16 or “public optional” 18 to another status, as indicated by arrows 81, 83, 85, 87, 89, and 91. It may also happen that a donor does not pay the deposit and/or a periodic premium, so the provider of the bank may decide to change the status of a sample having the status “private” 14 or “public optional” 18 to the status “public” 18, or to the status “discard” 64.

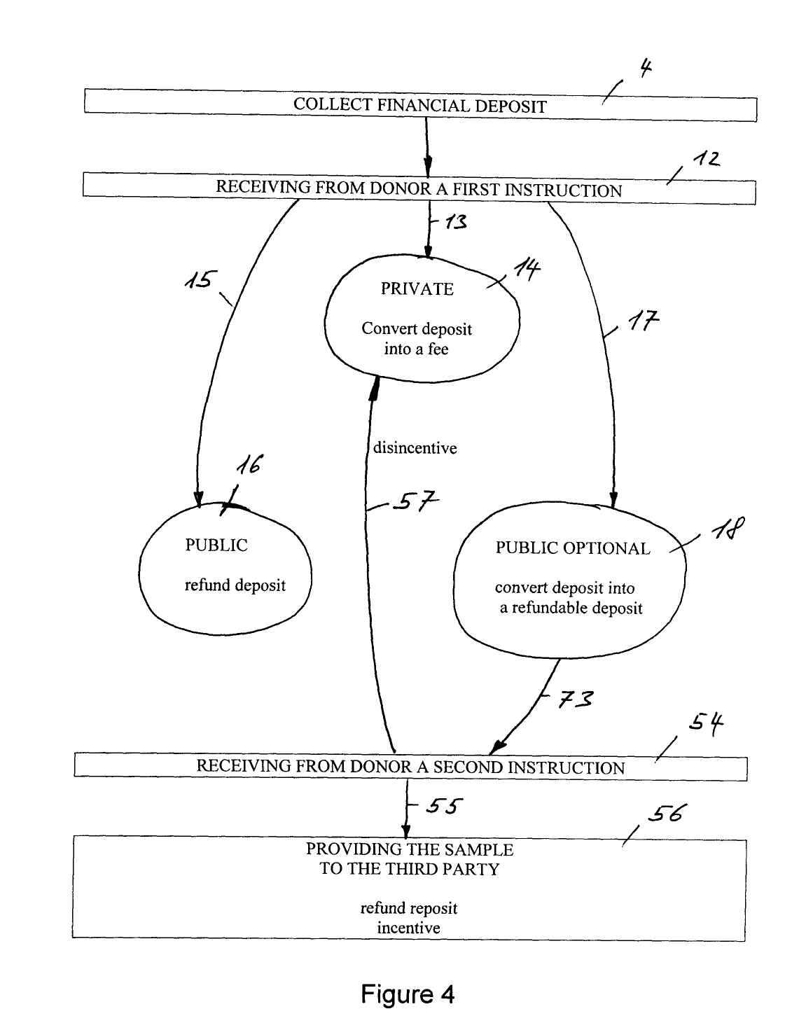

In a preferred method a deposit is collected and the status of the deposit may change depending on the status of the sample. FIG. 4 shows a status diagram of various statuses a deposit may have. The financial deposit is collected in step 4. After receiving from the donor the first instruction in step 12, the status of the sample is decided and, as shown in FIG. 4, also the status of the deposit. If the status of the sample becomes “public” 16 (via 15), the deposit is refunded to the donor. If the status of the sample becomes “private” 14 (via 13), the deposit is retained and converted into a fee. If the status of the sample becomes “public optional” 18 (via 17), the deposit is converted into a refundable deposit.

After a third party request and the selected sample having the status “public optional” 18, the method disclosed in FIG. 2 applies. Depending on the second instruction 54 received from the donor, as shown in FIG. 4, the sample having the status “public optional” will, as indicated by arrows 73, 55 and 57, either be provided to the third party in step 56, or the sample will have the status “private” 14. The donor will receive a disincentive if he selects the status “private” 14, at least in so far that the refundable deposit is converted into a fee. The donor will receive an incentive if he agrees to provide the sample to the third party in step 56, at least in so far that the deposit will be refunded in step 56. Preferably the donor receives an additional incentive, for example an amount of money.

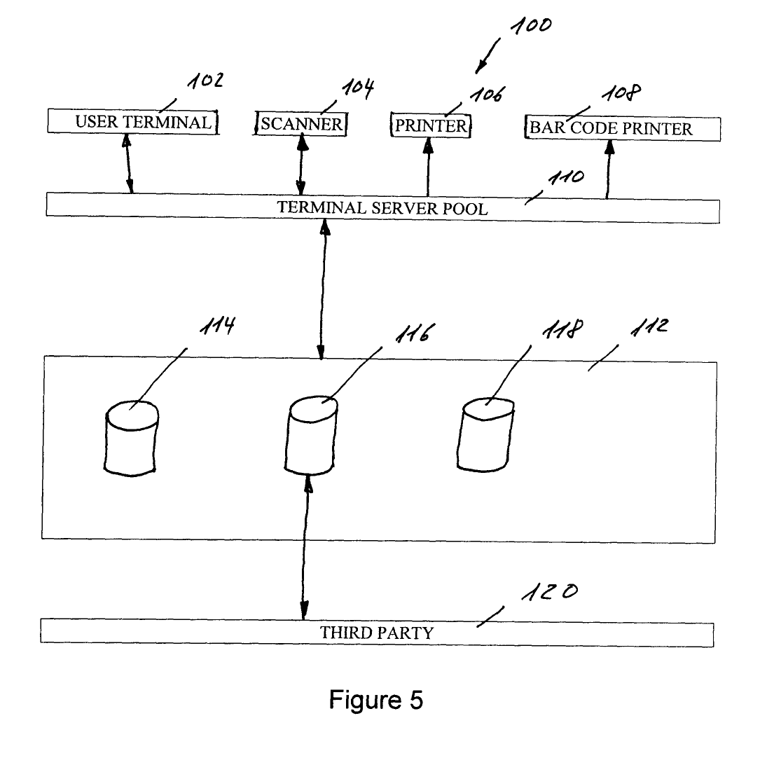

FIG. 5 shows a system 100 for providing samples of cells or tissue in accordance with one embodiment of the invention. The system 100 comprises user terminals 102, scanners 104, printer 106, bar code printers 108, and a terminal server pool 110, associated with a data storage device 112 configured to maintain information in a database such as a cell or tissue donor database 114, a sample identifier database 116, and a financial deposit status database 118. The database 116 also comprises typing information of the sample, such as HLA typing information. The system 100 allows managing all data to run the method according to the invention. Third parties 120 may access at least the sample identifier database 116.

The data storage device 100 is configured to maintain information in a database such that a cell or tissue donor information 114 is associated with a cell or tissue sample identifier database 116, the sample identifier database 116 is associated with a registry status, the registry status being selected from the group consisting of undetermined status, relinquished status, a private registry status, a public optional registry status, and a public registry status, and a deposit status database 118 of a financial deposit selected from the group consisting of a refundable deposit and a non-refundable deposit is associated with the registry status.

Thus, specific methods of storing a sample of cells or tissue in a cell or tissue bank have been disclosed. It should be apparent, however, to those skilled in the art that many more modifications besides those already described are possible without departing from the inventive concepts herein. The inventive subject matter, therefore, is not to be restricted except in the spirit of the present disclosure. Moreover, in interpreting the specification and contemplated claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Furthermore, where a definition or use of a term in a reference, which is incorporated by reference herein, is inconsistent or contrary to the definition of that term provided herein, the definition of that term provided herein applies and the definition of that term in the reference does not apply.

1. A method of storing a sample of cells or tissue in a cell or tissue bank, comprising:
receiving the sample from a donor, and associating the sample in response to a first donor instruction with one of a private registry, a public registry or a public optional registry; and if the donor opts for the public optional registry
receiving from the donor, in response to a third party request for the sample, a second instruction to either switch association from the public optional registry to the private registry or to allow providing the sample to the third party requestor.

2. The method of claim 1 wherein the donor is provided an incentive when the sample is provided to the third party requestor; and
wherein the donor is provided a disincentive when the donor switches association from the public optional registry to the private registry.

3. The method of claim 1 wherein the received sample is subdivided into at least two parts, and wherein one part of the subdivided sample is associated to the private registry.

4. The method of claim 1 wherein the first donor instruction is requested after the step of receiving the sample and before a step of typing the sample.

5. The method of claim 1 wherein the first donor instruction is requested within four weeks of receiving the sample.

6. The method of claim 1 further comprising a step of collecting a financial deposit from the donor.

7. The method of claim 6 wherein the financial deposit is collected before receiving the sample.

8. The method of claim 6 wherein the financial deposit is converted to a fee when the donor opts in the first instruction for association with the private registry.

9. The method of claim 6 wherein the financial deposit is converted to a refundable deposit when the donor opts in the first instruction for association with the public optional registry.

10. The method of claim 6 wherein the financial deposit is returned to the donor when the donor opts in the first instruction for association with the public registry so that the sample is donated to the bank.

11. The method of claim 9 wherein the refundable deposit is returned to the donor if the donor allows providing the sample to the third party requestor.

12. The method of claim 1 further comprising a step of providing financial compensation to the donor when the sample is provided to a third party requestor.

13. The method of claim 9 wherein the refundable deposit is converted to a fee when the donor opts in the second instruction or in a voluntary decision to change registry status from public optional registry to private registry.

14. The method of claim 2 wherein the step of providing an incentive comprises conversion of a financial deposit into a refundable, optionally interest-bearing deposit, and wherein the step of providing a disincentive is conversion of a financial deposit into a non-refundable fee.

15. The method of claim 1 further comprising a step of HLA typing of the sample upon association of the sample with the public registry or public optional registry.

16. The method of claim 1 further comprising the step of collecting a periodic premium.

17. The method of claim 1 further comprising a step of storing the sample in a plurality of aliquots.

18. The method of claim 1 further comprising a step of in vitro expanding, differentiation, or conversion, of at least a subpopulation of cells in the sample.

19. The method of claim 10, further receiving from the donor, if his sample is still available in the public registry, an instruction to either switch association from the public registry to the public optional registry or the private registry, and collecting a financial deposit or a fee.

20. A data storage device configured to maintain information in a database such that (a) a cell or tissue donor information is associated with a cell or tissue sample identifier, (b) the sample identifier is associated with a registry status selected from the group consisting of undetermined status, relinquished status, a private registry status, a public optional registry status, and a public registry status.

21. The data storage device of claim 20, wherein a deposit status of a financial deposit selected from the group consisting of a refundable deposit and a non-refundable deposit is associated with the registry status.

22. The data storage device of claim 20 wherein the database is further configured to allow access by a third party to the sample identifier when the registry status is the public optional or the public registry status.

23. The data storage device of claim 22 wherein the database is further configured to store HLA typing information for a sample having public optional or public registry status.

24. The data storage device of claim 20 wherein the database is configured such that the undetermined registry status is allowed for only a predetermined period of less than 30 days.

25. The data storage device of claim 20 wherein the database is configured such that the relinquished registry status is associated with the sample identifier when the deposit status is the refundable deposit and when the donor information indicates cancellation of a contract.

27. The data storage device of claim 20 wherein the database is further configured to associate periodic premium payment status with the donor information.
